(19) United States
(12) Patent Application Publication
(10) Pub. No.: US 2024/0325639 A1
(43) Pub. Date: Oct. 3, 2024

(54) BLOOD GLUCOSE ESTIMATION BASED ON RATE OF CHANGE INFORMATION

(71) Applicant: MEDTRONIC MINIMED, INC., Northridge, CA (US)

(72) Inventors: Yuxiang Zhong, Arcadia, CA (US); Arthur Mikhno, Princeton, NJ (US); Michael P. Stone, Long Beach, CA (US)

(21) Appl. No.: 18/600,507

(22) Filed: Mar. 8, 2024

Related U.S. Application Data
(60) Provisional application No. 63/493,997, filed on Apr. 3, 2023.

(51) Int. Cl. A61M 5/172; G06N 5/022; G16H 40/63

(52) U.S. Cl. CPC A61M 5/1723; G06N 5/022; G16H 40/63; A61M 2205/502; A61M 2230/201

(57) ABSTRACT

Disclosed herein are techniques related to blood glucose estimation based on rate of change information. In some embodiments, the techniques may involve obtaining a sensor glucose value. The techniques may also involve calculating a sensor glucose rate of change based on the sensor glucose value. The techniques may further involve determining an estimated glucose bias value corresponding to the sensor glucose rate of change. The determining may be based on applying, to the sensor glucose rate of change, a predictive model for estimating glucose bias values corresponding to differences between blood glucose measurements and sensor glucose values. Additionally, the techniques may involve adjusting the sensor glucose value based on accounting for the estimated glucose bias value.

### CROSS-REFERENCE TO RELATED APPLICATIONS

This application claims the benefit of priority to U.S. Provisional Application No. 63/493,997, filed Apr. 3, 2023, which is incorporated herein by reference in its entirety.

### TECHNICAL FIELD

The present disclosure relates generally to the medical arts and more particularly to blood glucose estimation based on rate of change information.

### BACKGROUND

A person (e.g., a patient or a user of a diabetes management device) may use insulin therapy to manage type I or type II diabetes. Insulin therapy may include use of insulin infusion systems for delivering or dispensing insulin. An insulin infusion system may include an infusion device which typically includes a small motor and drive train components configured to deliver insulin from a reservoir into the body of a person, e.g., via a percutaneous needle or a cannula placed in the subcutaneous tissue. Insulin infusion systems may facilitate management of diabetes.

### SUMMARY

Disclosed herein are techniques related to blood glucose estimation based on rate of change information. The techniques may be practiced in a variety of ways, such as using a processor-implemented method; a system comprising one or more processors and one or more processor-readable media; and/or one or more processor-readable media (e.g., non-transitory processor-readable media).

In some embodiments, the techniques may involve obtaining a sensor glucose value. The techniques may also involve calculating a sensor glucose rate of change based on the sensor glucose value. The techniques may further involve determining an estimated glucose bias value corresponding to the sensor glucose rate of change. The determining may be based on applying, to the sensor glucose rate of change, a predictive model for estimating glucose bias values corresponding to differences between actual blood glucose levels and contemporaneous sensor glucose values. Additionally, the techniques may involve adjusting the sensor glucose value based on accounting for the estimated glucose bias value.

### BRIEF DESCRIPTION OF THE DRAWINGS

The above and other aspects and features of the disclosure will become more apparent in view of the following detailed description when taken in conjunction with the accompanying drawings wherein like reference numerals identify like elements.

FIG. 1 is a diagram of an example therapy delivery system, in accordance with aspects of the present disclosure.

FIG. 2 is a diagram of an example insulin delivery device, in accordance with aspects of the present disclosure.

FIG. 3 is a flow diagram illustrating example techniques for estimating blood glucose with increased accuracy, in accordance with aspects of the present disclosure.

FIG. 4 is a block diagram illustrating an example data store including glucose-related data and contextual data, in accordance with aspects of the present disclosure.

FIG. 5 is a flow diagram illustrating example bucketization techniques for generating a bias value estimation model, in accordance with aspects of the present disclosure.

FIG. 6 is a block diagram illustrating an example predictive model for estimating bias values, in accordance with aspects of the present disclosure.

FIG. 7 is a flow diagram illustrating example techniques for estimating bias values based on applying a predictive model, in accordance with aspects of the present disclosure.

### DETAILED DESCRIPTION

Techniques related to estimating blood glucose with increased accuracy are described. Blood glucose levels can be determined or estimated using glucometers and/or continuous glucose monitors (CGMs). Using a glucometer typically involves applying a drop (or more) of a patient's blood to a “test strip,” which is analyzed to determine the patient's blood glucose measurement. This blood glucose measurement can be used for a variety of purposes, such as to determine an insulin delivery amount or to calibrate a CGM.

A CGM is typically worn on the skin (e.g., stomach, arm, etc.) and includes a sensor that measures glucose levels in a bodily fluid other than blood. For example, many CGMs include sensors that measure glucose levels within the surrounding interstitial fluid. Although CGMs can frequently (e.g., every five minutes) measure glucose levels without blood samples inconveniently taken with corresponding frequency, the glucose measurements taken by CGMs often differ from blood glucose measurements (which are considered to be the “gold standard” in terms of accuracy). To account for this deviation from blood glucose measurements, glucose measurements taken by CGMs (e.g., interstitial glucose measurements) can undergo a calibration process to convert such measurements into approximations of blood glucose measurements. Such approximations are referred to herein as “sensor glucose (SG) values,” which are often relied upon (e.g., periodically displayed and/or analyzed) as if they were equivalent to blood glucose measurements.

While CGMs can generate a lot of SG values estimating a patient's blood glucose levels, these estimated blood glucose levels can “lag” behind actual blood glucose levels, thereby creating a difference or “bias” between the patient's actual blood glucose level and the estimated blood glucose level that is provided as an SG value. This is especially noticeable when the patient is experiencing a high rate of change in blood glucose level. For example, an interstitial sensor glucose level reading can take as much as 15 to 30 minutes to “catch up” with a blood glucose level reading if the patient's glucose levels are changing at a high rate (e.g., more than three milligrams/deciliter per minute (mg/dl/min)). Given the potential lag between sensor glucose levels and blood glucose levels, a patient may rely on a glucometer for a blood glucose reading prior to administering insulin.

To address SG bias and its ramifications (e.g., for insulin administration), disclosed herein are techniques related to blood glucose (BG) estimation based on SG rate of change. The techniques may involve generating a predictive model that correlates at least SG rate of change information with SG bias estimations. Additionally or alternatively, the techniques may involve estimating SG bias based on application of the predictive model to at least SG rate of change information.

The predictive model may be generated based on collecting, aggregating, recording (e.g., by periodically storing records), and/or analyzing a patient's historical data, which can include a variety of information. Non-limiting examples of such information include:
BG measurements (e.g., measured by a glucometer using blood from a finger prick);
SG values (e.g., generated by a CGM based on interstitial glucose measurements);
bias values corresponding to differences between actual blood glucose levels and contemporaneous SG values;
SG rate of change values (e.g., calculated by taking the change in SG value relative to an immediately preceding SG value and dividing this change by the amount of time elapsed since the immediately preceding SG value was generated); and
contextual information (e.g., time of day information, day of week information, sensor age information, sensor condition descriptors, indications of any active sensor alerts, and/or other information indicative of contexts in which SG values were generated).
   The historical data may be analyzed and/or otherwise processed to generate a predictive model that correlates SG values, SG rate of change values, and/or contextual information with a plurality of candidate bias values. Each candidate bias value may correspond to a respective probability value. For example, the predictive model may correlate a particular combination of SG value, SG rate of change, and contextual information with candidate bias values of −5 mg/dl (88%), −10 mg/dl (10%), −15 mg/dl (2%), and −20 mg/dl (0%). Thus, the final estimated bias value may be selected from the candidate bias values based on probability (e.g., the candidate bias value with the highest probability value may be selected).

The predictive model may be used to estimate SG bias for a patient based on the patient's SG value, SG rate of change value, and/or contextual information. For example, if a patient's sensor glucose level is 126 mg/dl (as determined by a CGM that has been worn for 4 days, is losing sensitivity, and has an active alert indicating a sensor error) and rising by 1 mg/dl/min at 3 pm on a Monday, the predictive model can be applied to some or all of the foregoing information to determine a corresponding SG bias estimation. This bias estimation can then be used to adjust (e.g., added to or subtracted from) the patient's sensor glucose level to determine an adjusted sensor glucose level that is a more accurate blood glucose estimation.

There are many beneficial uses for SG values that have been adjusted to account for estimated bias. Such SG values can be presented (e.g., displayed) to a patient to improve glucose management by better informing the patient with more accurate blood glucose estimations, thereby enabling the patient to make lifestyle adjustments (e.g., modifying diet and/or exercise regimens). Additionally or alternatively, such SG values can help avoid insulin underdelivery or overdelivery by facilitating calculation of appropriate insulin dosages based on more accurate blood glucose estimations. Notably, such SG values can be derived without reliance on contemporaneous blood glucose measurements that are obtained by requiring patients to endure the inconvenience of finger pricks. As used herein, SG values and BG levels/measurements are considered to be contemporaneous even if the SG values and the BG levels/measurements are separated in time by a lag.

The present disclosure is described primarily with respect to glucose management. However, one of ordinary skill in the art will recognize that the disclosed techniques can be used to account for biases in the estimation of other analyte values as well, especially where the analyte value being estimated is rapidly changing and there is a lag between the actual value and the value determined by a monitoring device (i.e., sensed data).

Discussions utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other non-transitory information storage media that may store instructions to perform operations and/or processes.

Unless explicitly stated, the methods described herein are not constrained to a particular order or sequence. Additionally, some of the described methods or elements thereof can occur or be performed simultaneously or concurrently.

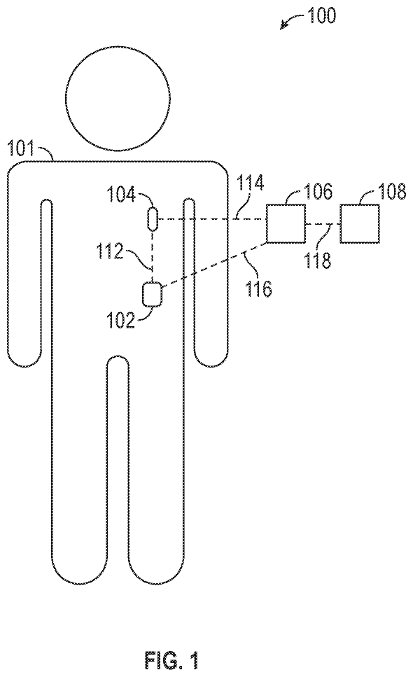

FIG. 1 depicts an example therapy delivery system 100 for a person 101. System 100 may be an insulin delivery system. The depicted therapy delivery system 100 includes a delivery device 102, a monitoring device 104, a computing device 106, and an optional remote or cloud computing system 108. The delivery device 102, the monitoring device 104, and the computing device 106 may be embodied in various ways, including being disposed in one or more device housings. For example, in some embodiments, all of the devices 102-106 may be disposed in a single device housing. In some embodiments, each of the devices 102-106 may be disposed in a separate device housing. In some embodiments, two or more of the devices 102-106 may be disposed in the same device housing, and/or a single device 102, 104, or 106 may have two or more parts that are disposed in two or more housings. Such embodiments, and combinations thereof, are contemplated to be within the scope of the present disclosure.

FIG. 1 also depicts communications links 112-118. The communications links 112-118 may each be a wired connection and/or a wireless connection. In the case where two devices are located in the same device housing, the communication link may include, for example, wires, cables, and/or communication buses on a printed circuit board, among other things. In the case where two devices are separated from each other in different device housings, the communication links may be wired and/or wireless connections. Wired connections may include, without limitation, an Ethernet connection, a USB connection, and/or another type of physical connection. Wireless connections may include, without limitation, a cellular connection, a Wi-Fi connection, a Bluetooth® connection, a mesh network connection, and/or another type of connection using a wireless communication protocol. Some embodiments of the communication links 112-118 may use direct connections, such as Bluetooth® connections, and/or may use connections that route through one or more networks or network devices (not shown), such as an Ethernet network, a Wi-Fi network, a cellular network, a satellite network, an intranet, an extranet, the Internet, and/or the Internet backbone, among other types of networks. Various combinations of wired and/or wireless connections may be used for the communication links 112-118.

Aspects of the therapy delivery system 100 are described below. Further aspects and details may be described in U.S. Pat. Nos. 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; 7,621,893; and 9,364,609. The entire contents of each of the foregoing United States Patents are hereby incorporated by reference herein.

The delivery device 102 is configured to deliver a therapeutic substance (e.g., insulin) to a person 101. The delivery device 102 may be secured to the person 101 (e.g., to the body or clothing of the person 101) or may be implanted on or in the body of the person 101. In some embodiments, the delivery device 102 may include a reservoir, an actuator, a delivery mechanism, and a cannula (not shown). The reservoir may be configured to store an amount of the therapeutic substance. In some embodiments, the reservoir may be refillable or replaceable. The actuator may be configured to drive the delivery mechanism. In some examples, the actuator may include a motor, such as an electric motor. The delivery mechanism may be configured to move the therapeutic substance from the reservoir through the cannula. In some examples, the delivery mechanism may include a pump and/or a plunger. The cannula may facilitate a fluidic connection between the reservoir and the body of the person 101. The cannula and/or a needle may facilitate delivery of the therapeutic substance to a tissue layer, vein, or body cavity of the person 101. During operation, the actuator, in response to a signal (e.g., a command signal), may drive the delivery mechanism, thereby causing the therapeutic substance to move from the reservoir, through the cannula, and into the body of the person 101.

The components of the delivery device 102 described above are merely provided as examples. The delivery device 102 may include other components, such as, without limitation, a power supply, a communication transceiver, computing resources, and/or user interfaces, among other things. Persons skilled in the art will recognize various implementations of the delivery device 102 and the components of such implementations. All such implementations and components are contemplated to be within the scope of the present disclosure.

With continuing reference to FIG. 1, the monitoring device 104 is configured to detect a physiological condition (e.g., a glucose concentration level) of the person 101 and may also be configured to detect other things. The monitoring device 104 may be secured to the body of the person 101 (e.g., to the skin of person 101 via an adhesive) and/or may be at least partially implanted into the body of the person 101. Depending on the particular location or configuration, the monitoring device 104 may be in contact with biological matter (e.g., interstitial fluid and/or blood) of the person 101.

The monitoring device 104 includes one or more sensors (not shown), such as, without limitation, electrochemical sensors, electrical sensors, and/or optical sensors. As persons skilled in the art will understand, an electrochemical sensor may be configured to respond to the interaction or binding of a biological marker to a substrate by generating an electrical signal based on a potential, conductance, and/or impedance of the substrate. The substrate may include a material selected to interact with a particular biomarker, such as glucose. The potential, conductance, and/or impedance may be proportional to a concentration of the particular biomarker. In the case of electrical sensors, and as persons skilled in the art will understand, an electrical sensor may be configured to respond to an electrical biosignal by generating an electrical signal based on an amplitude, frequency, and/or phase of the electrical biosignal. The electrical biosignal may include a change in electric current produced by the sum of an electrical potential difference across a tissue, such as the nervous system, of the person 101. In some embodiments, the electrical biosignal may include portions of a potential change produced by the heart of the person 101 over time, e.g., recorded as an electrocardiogram, that are indicative of a glucose level of the person 101. In the case of optical sensors, as persons skilled in the art will understand, an optical sensor may be configured to respond to the interaction or binding of a biological marker to a substrate by generating an electrical signal based on change in luminance of the substrate. For example, the substrate may include a material selected to fluoresce in response to contact with a selected biomarker, such as glucose. The fluorescence may be proportional to a concentration of the selected biomarker.

In some embodiments, the monitoring device 104 may include other types of sensors that may be worn, carried, or coupled to the person 101 to measure activity of the person 101 that may influence the glucose levels or glycemic response of the person 101. As an example, the sensors may include an acceleration sensor configured to detect an acceleration of the person 101 or a portion of the person 101, such as the person's hands or fect. The acceleration (or lack thereof) may be indicative of exercise, sleep, or food/beverage consumption activity of the person 101, which may influence the glycemic response of the person 101. In some embodiments, the sensors may include heart rate and/or body temperature sensors, which may indicate an amount of physical exertion experienced by the person 101. In some embodiments, the sensors may include a GPS receiver which detects GPS signals to determine a location of the person 101.

The sensors described above are merely provided as examples. Other sensors or types of sensors for monitoring physiological condition, activity, and/or location, among other things, will be recognized by persons skilled in the art and are contemplated to be within the scope of the present disclosure. For any sensor, the signal provided by a sensor shall be referred to as a “sensor signal.”

The monitoring device 104 may include components and/or circuitry configured to pre-process sensor signals. Pre-processing may include, without limitation, amplification, filtering, attenuation, scaling, isolation, normalization, transformation, sampling, and/or analog-to-digital conversion, among other things. Persons skilled in the art will recognize various implementations for such pre-processing, including, without limitation, implementations using processors, controllers, ASICS, integrated circuits, hardware, firmware, programmable logic devices, and/or machine-executable instructions, among others. The types of pre-processing and their implementations are merely provided as examples. Other types of pre-processing and implementations are contemplated to be within the scope of the present disclosure. In some embodiments, the monitoring device 104 may not perform pre-processing.

As used herein, the term “sensed data” shall mean and include the information represented by a sensor signal or by a pre-processed sensor signal. In some embodiments, sensed data may include glucose levels in a person 101, acceleration of a part of the person 101, heart rate of the person 101, temperature of the person 101, and/or geolocation (e.g., GPS location) of the person 101, among other things. The monitoring device 104 may communicate sensed data to the delivery device 102 via communication link 112 and/or to the computing device 106 via communication link 114. Use of sensed data by the delivery device 102 and/or by the computing device 106 will be described later herein.

The computing device 106 provides processing capabilities and may be implemented in various ways. In some embodiments, the computing device 106 may be a consumer device, such as a smartphone, a computerized wearable device (e.g., a smartwatch), a tablet computer, a laptop computer, or a desktop computer, among others, or may be a special purpose device (e.g., a portable control device) provided by, for example, the manufacturer of the delivery device 102. In some embodiments, the computing device 106 may be “processing circuitry” (defined below) that is integrated with another device, such as the delivery device 102. In some embodiments, the computing device 106 may be secured to the person 101 (e.g., to the body or clothing of person 101), may be at least partially implanted into the body of person 101, and/or may be held by the person 101.

For each of the embodiments of the computing device 106, the computing device 106 may include various types of logic circuitry, including, but not limited to, microprocessors, controllers, digital signal processors (DSPs), application specific integrated circuits (ASICs), field-programmable gate arrays (FPGAs), central processing units (CPU), graphics processing units (GPU), programmable logic devices, memory (e.g., random access memory, volatile memory, non-volatile memory, etc.), or other discrete or integrated logic circuitry, as well as combinations of such components. The term “processing circuitry” may generally refer to any of the foregoing logic circuitry, alone or in combination with other logic circuitry, or any other circuitry for performing computations.

Aspects of the delivery device 102, the monitoring device 104, and the computing device 106 have been described above. One or more of the devices 102-106 may include a user interface (not shown) that presents information to the person 101 and/or receives information from the person 101. The user interface may include a graphical user interface (GUI), a display device, a keyboard, a touchscreen, a speaker, a microphone, a vibration motor, buttons, switches, and/or other types of user interfaces. Persons skilled in the art will recognize various types of user interfaces that may be used, and all such user interfaces are contemplated to be within the scope of the present disclosure. For example, where the computing device 106 is a consumer device such as a smart phone, tablet computer, laptop computer, or the like, the user interfaces would include a display device, a physical and/or virtual keyboard, and/or audio speakers provided by such consumer devices, among other things. In some embodiments, a user interface may notify the person 101 of sensed data (e.g., glucose level) and/or insulin delivery data (e.g., rates of historic, current, or future insulin delivery) and may present alerts to the person 101. In some embodiments, a user interface may receive inputs from the person 101, which may include, for example, a requested change in insulin delivery and/or a meal indication, among other things. The descriptions and embodiments above regarding user interfaces are merely provided as examples, and other types and other uses of user interfaces are contemplated to be within the scope of the present disclosure.

The following describes communications between the devices 102-106 and cooperation between the devices 102-106 with respect to insulin delivery. As depicted in FIG. 1, and as mentioned above, the devices 102-106 may communicate with each other via communication links 112-116. In some embodiments, the computing device 106 may control operation of the delivery device 102 and/or the monitoring device 104. For example, the computing device 106 may generate one or more signals (e.g., a command signal) that cause the delivery device 102 to deliver insulin to the person 101, e.g., as a basal dosage and/or a bolus dosage. In some embodiments, the computing device 106 may receive data associated with insulin delivery (e.g., insulin delivery data) from the delivery device 102 and/or receive sensed data (e.g., glucose levels) from the monitoring device 104 and may perform computations based on the insulin delivery data, the sensed data, and/or other data to control the delivery device 102. Insulin delivery data may include, but is not limited to, a type of insulin being delivered, historical insulin delivery rates and/or amounts, current insulin delivery rate and/or amount, and/or user input affecting insulin delivery. As persons skilled in the art will understand, in a closed-loop operating mode, computing device 106 may communicate dosage commands to the delivery device 102 based on a difference between a current glucose level in the body of the person 101 (e.g., received from the monitoring device 104) and a target glucose level (e.g., determined by the computing device 106). The dosage commands may indicate an amount of insulin to be delivered and/or a rate of insulin delivery and may regulate the current glucose level toward the target glucose level. Examples of closed-loop operations for insulin infusion systems are described in U.S. Pat. Nos. 6,088,608, 6,119,028, 6,589,229, 6,740,072, 6,827,702, 7,323,142, and 7,402,153, and in United States Patent Application Publication Nos.: 2014/0066887 and 2014/0066889. The entire contents of each of the foregoing patents and publications are hereby incorporated by reference herein.

With continuing reference to FIG. 1, the remote or cloud computing system 108 may be a proprietary remote/cloud computing system or a commercial cloud computing system including one or more server computing devices. The remote/cloud computing system 108 may provide additional computing resources on-demand as needed when the computing resources of a client computing device (e.g., the computing device 106) are not sufficient. The computing device 106 and the remote/cloud computing system 108 may communicate with each other through a communication link 118, which may traverse one or more communication networks (not shown). The delivery device 102 may communicate with the remote/cloud computing system 108 by way of the computing device 106 via communication links 116 and 118 or directly via other communication links (not shown). The monitoring device 104 may communicate with the remote/cloud computing system 108 by way of the computing device 106 via communication links 114 and 118 or directly via other communication links (not shown). The communication networks may include, without limitation, an Ethernet network, Bluetooth® network, Wi-Fi network, a cellular network, a satellite network, an intranet, an extranet, the Internet, and/or the Internet backbone, among other types of networks. Persons skilled in the art will recognize implementations for the remote/cloud computing system 108 and how to interface with such systems through various types of networks. For example, the remote/cloud computing system 108 may include an array of processing circuitry (defined above) and may execute machine-readable instructions. Such implementations, interfaces, and networks are contemplated to be within the scope of the present disclosure.

An example therapy delivery system has been described above. For convenience, the description below may primarily refer to an insulin delivery system as an example of the therapy delivery system. However, it is intended that any aspect, embodiment, or description relating to an insulin delivery system shall be applicable to a therapy delivery system which delivers a therapy other than insulin.

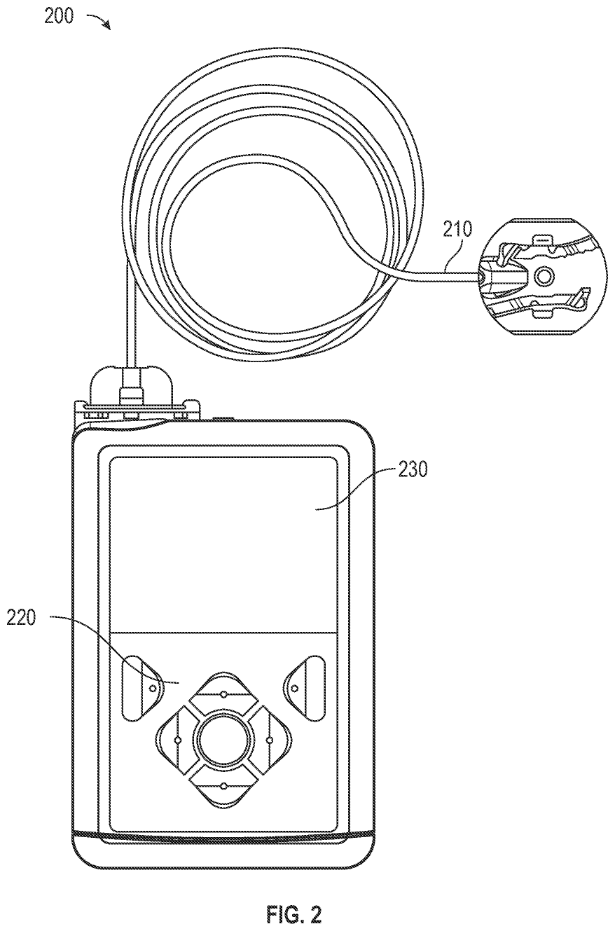

In some embodiments, therapy may be affected based on communicating a therapy determination toward a therapy delivery device. A non-limiting example of such a device is described below in connection with FIG. 2.

As mentioned above, therapy determinations may be communicated toward an insulin delivery device 200 (e.g., from a cloud computing system 108 via an intermediary computing device 106 communicatively coupled to the device 200 or from the computing device 106 directly to the device 200). In device 200, insulin delivery may be performed based on internal communication between a central computing module (e.g., including a microcontroller) and an insulin delivery module (e.g., including a motor, a gear train, a reservoir, and/or a plunger). For instance, insulin delivery may be caused by the central computing module communicating a delivery command in the form of an electrical signal that travels via a communication fabric to the insulin delivery module. The central computing module may also be configured to communicate (e.g., via a transceiver) with a computing device (e.g., 106, FIG. 1) communicatively coupled to a remote or cloud computing system (e.g., 108, FIG. 1).

The insulin delivery device 200 can provide fast-acting insulin through a small tube 210 configured for fluidic connection with a cannula (not shown) that is inserted subcutaneously. The device 200 can deliver at least two types of dosages—a basal dosage, which can be delivered periodically (e.g., every five minutes) in tiny amounts throughout the day and night, and a bolus dosage to cover an increase in blood glucose from meals and/or to otherwise correct high blood glucose levels. The depicted insulin delivery device 200 includes a user interface having button elements 220 that can be manipulated to administer a bolus dosage of insulin, to change therapy settings, to change user preferences, to select display features, and the like. The insulin delivery device 200 also includes a display device 230 that can be used to present various types of information or data to the user. In accordance with aspects of the present disclosure, a user of the insulin delivery device 200 may use the button elements 220 to input certain event data (e.g., event type, event start time, event details, etc.), and the user inputs can be confirmed using the display device 230. The depicted insulin delivery device 200 of FIG. 2 is merely provided by way of example, and other types of insulin delivery devices and other techniques different from those described above are contemplated to be within the scope of the present disclosure.

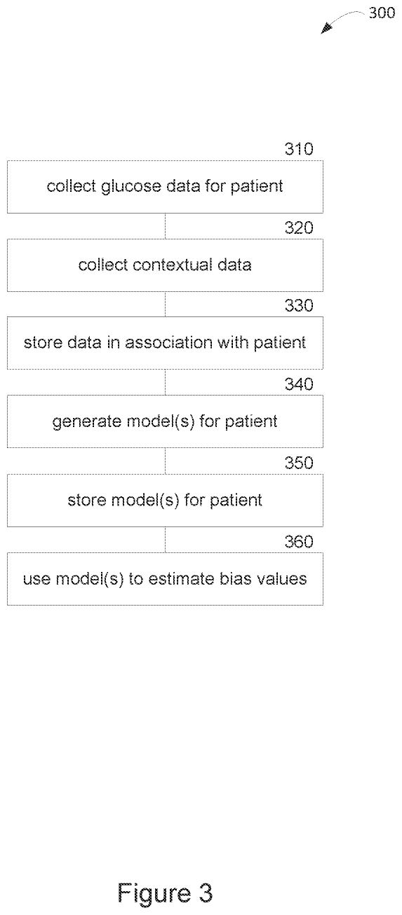

FIG. 3 is a flow diagram illustrating example techniques 300 for estimating blood glucose with increased accuracy. Techniques 300 may be performed by one or more devices (e.g., delivery device 102, monitoring device 104, computing device 106, and/or computing system 108). For example, blocks 310-340 may be performed by computing system 108, and blocks 350-360 may be performed by monitoring device 104.

At block 310, glucose data is collected (e.g., periodically) for a patient. The glucose data may include a blood glucose measurement (e.g., provided by a glucometer or a person using a glucometer) and/or a sensor glucose value (e.g., provided by a CGM). In some embodiments, the glucose data is processed (e.g., by one or more of devices 102-106 and/or computing system 108) to determine analytical data related to the glucose data. For example, a pair of successive SG values may be used to determine a sensor glucose rate of change, and a contemporaneous pair of BG and SG values may be used to determine a bias value corresponding to the difference between the BG value and SG value.

At block 320, contextual data is collected (e.g., from one or more of devices 102-106). The contextual data may be contemporaneous with the collected glucose data and may be indicative of a context in which a particular SG value was generated. Non-limiting examples of contextual data include information indicating the time of day, the day of the week, the time since the patient last ate or exercised, the patient's location, the patient's age, the patient's weight, the health of the CGM (e.g., whether device 104 is warming up, stabilized, or losing sensitivity), sensor age (e.g., how long a sensor of the monitoring device 104 has been in use), the CGM's battery levels, and/or the presence of any active alerts (e.g., an alert indicating an unresolved sensor error) for the CGM.

At block 330, collected information is stored in association with the patient, such as in a record (tuple) of a database. In some embodiments, the collected information may also be stored with analytical data related to the glucose data. For example, each record may correspond to a snapshot of the patient's glucose-related data, including a bias value, at a particular point in time and may further include contextual information that can affect the bias value. These records can serve as historical data that enables estimation of bias values for future SG values of the patient.

At block 340, based on the data stored in association with the patient, one or more patient-specific models are generated for estimating bias values. This can be achieved in a variety of ways.

In some embodiments, “bucketization” is used to generate the one or more models. For example, the one or more models may include an n-dimensional model that is constructed by selecting an attribute (e.g., SG value) from the patient's database records, splitting the records into different buckets (“bucketizing”) based on the range of values corresponding to the attribute (e.g., SG values of 240-400 mg/dL, SG values of 180-240 mg/dL, and so on), and further bucketizing the data in each bucket into sub-buckets based on another attribute (e.g., rate of change). This process can be repeated until some termination condition is reached for some or all of the buckets, such as the generation of at least a predetermined number of buckets, an average number of records per bucket (including sub-buckets) has been reached, and so on. In some cases, further bucketization may not be performed for buckets that include fewer than a predetermined number of records (e.g., 5, 10, 20).

An average bias value can be calculated for each bucket based on the records in each bucket. Alternatively, a probability may be assigned to each bias value in a bucket based on the number of records in that bucket with that bias value.

In some cases, attributes are selected for bucketization according to a predetermined list or order. In some other cases, attributes are randomly selected.

In some embodiments, regression is used to generate the one or more models. For example, linear regression or polynomial regression may be performed on the stored data to generate a formula to estimate a bias value (dependent variable) based on one or more attribute values (one or more independent variables, such as rate of change).

In some embodiments, machine learning techniques and/or models are employed to generate the one or more models (e.g., classifiers) trained using the stored data. For example, training data may include feature vectors, and each feature vector may include attribute values of a respective database record. Each classifier may be any of a variety or combination of classifiers including neural networks (e.g., a fully-connected, convolutional, recurrent, autoencoder, or restricted Boltzmann machine), a support vector machine, a Bayesian classifier, and so on. When the classifier is a deep neural network, the training results in a set of weights for the activation functions of the deep neural network.

Adaptive boosting is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates more and more on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, cost function, and search algorithm. The architecture defines the functional form relating the inputs to the outputs (in terms of network topology, unit connectivity, and activation functions). The search in weight space for a set of weights that minimizes the objective function is the training process. An example implementation may use a radial basis function network and a standard gradient descent as the search technique, a ridge basis function network with hyperparameter tuning, and so on.

In some cases, a subset of the stored data may be used to generate the model(s). For example, some of the stored data may be reserved “test data” for training the one or more models. This enables assessing the effectiveness of the one or more generated models, for example, by applying the one or more models to the test data to generate one or more scores, each score corresponding to how well a respective model estimates bias values. These assessments can be used to prune any models that do not score above a predetermined threshold. Additionally or alternatively, some of the stored data may be excluded from use in generating the model(s) based on age and/or other criteria. For instance, a linear or other weighting technique may be applied to the stored data so that older records are given less weight than newer records.

At block 350, the one or more patient-specific models are stored. In some embodiments, the one or more models may be locally stored on a device (e.g., any of devices 102-106) configured to use the one or more models to estimate bias values. In some other embodiments, the one or more models may be stored on a computing system (e.g., system 108) configured to use the one or models to estimate bias values that are communicated toward a remote device (e.g., any of devices 102-106).

At block 360, the model(s) are used to estimate bias values for SG values, each estimated bias value representing an estimated difference between a patient's actual blood glucose level and a contemporaneous SG value. An estimated bias value may be generated based on applying the model(s) to a patient's current SG value and/or contextual information indicative of a context in which the SG value was generated.

In some embodiments involving multiple models, one model may be selected for use in generating estimated bias values. For example, the selected model may have produced the best score when applied to test data.

In some other embodiments involving multiple models, multiple bias values may be generated using each of the multiple models, and a single bias value may be determined based on the multiple bias values. For example, the single bias value may correspond to an average, weighted average, mean, median, or mode of the multiples bias values.

Irrespective of the particular implementation used to estimate bias values, they can be used to estimate blood glucose levels with greater accuracy. For example, an estimated bias value can be added to or subtracted from a SG value to derive an adjusted SG value that more closely approximates a person's actual BG level. This adjusted SG value has a variety of beneficial uses. For example, the adjusted SG value can be presented (e.g., in an audio and/or visual format) to the person and/or used to calculate an insulin dosage that is communicated to an insulin delivery device or the person (e.g., a user of the insulin delivery device).

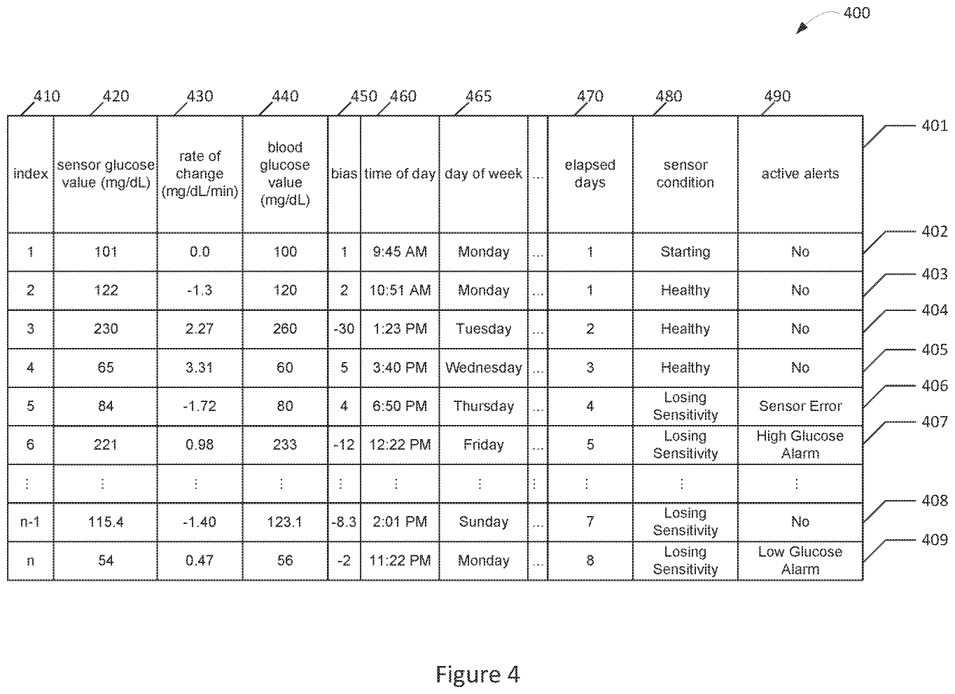

FIG. 4 is a block diagram illustrating an example data store 400 including glucose-related data and contextual data, in accordance with some embodiments of the disclosed technology. In this example, the data store 400 is represented as a table comprising a plurality of columns 410-490, each column representing a different attribute, and a plurality of rows 401-409, each row (e.g., tuple or feature vector) including a set of values for the attributes. Data store 400 includes an index column 410, a sensor glucose value column 420, a sensor glucose rate of change column 430, a blood glucose value column 440, a bias column 450, a time of day column 460, a day of week column 465, an elapsed days column 470, a sensor condition column 480, an active alerts column 490, etc. Each of rows 402-409 represents a separate set of attribute values representative of the patient's glycemic condition (in columns 420-450) and contextual information (in columns 460-490) at the time the patient's glycemic condition was estimated.

For example, row 404, which corresponds to index 3 in the data store 400, represents the patient's glycemic condition and contextual information at the time the SG value of column 420 was generated (e.g., by device 104). In this example, the patient had a sensor glucose value of 230 mg/dL, a SG rate of change of 2.27 mg/dL/min, a blood glucose level of 260 mg/dL, and a bias of −30 (mg/dL). In addition to this glycemic, row 404 includes contextual information indicating the SG value was calculated at 1:23 p.m. on a Tuesday by a monitoring device that had been operating for 2 days and was operating normally (in a “healthy” sensor condition and free of any alerts warning of an anomaly) when the SG value was calculated.

One skilled in the art will appreciate that while FIG. 4 provides an illustration that is easily comprehensible by a human reader, the actual information may be stored using different data structures and data organizations. Moreover, the information recorded in the data store may include fewer or additional attributes and may include rows with missing attribute values. For example, the data store may include additional attributes about the patient (e.g., height, weight, age, heart rate, time since last meal, and/or time since medication administered) and/or additional contextual information (e.g., month, temperature, and/or location).

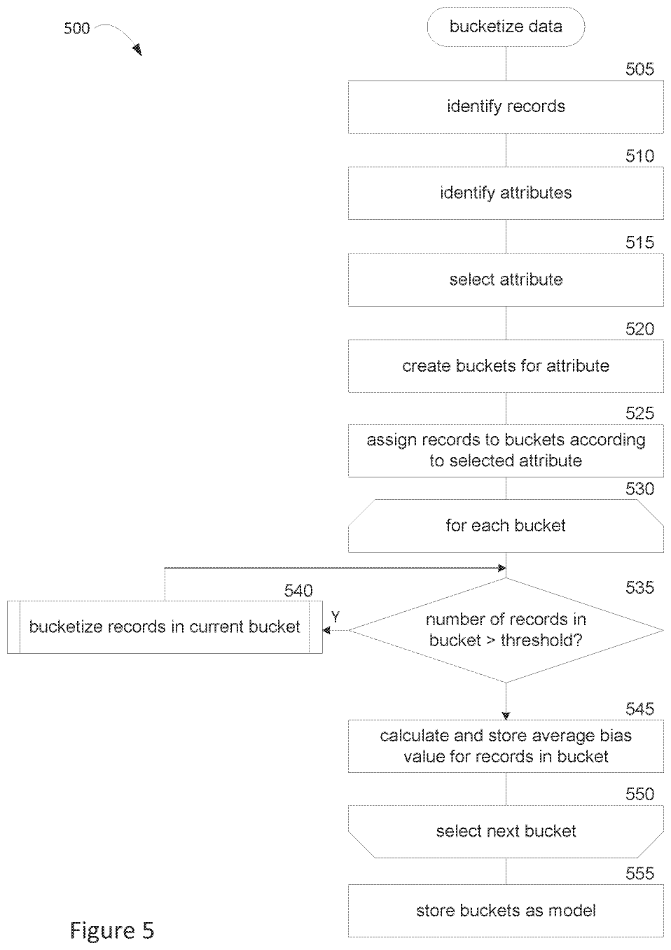

FIG. 5 is a flow diagram illustrating example bucketization techniques 500 for generating a bias value estimation model, in accordance with some embodiments of the disclosed technology. To generate a patient-specific model for estimating sensor glucose bias values, records may be sorted into individual buckets (and sub-buckets), each bucket representing a range of attribute values stored therein.

At block 505, records are identified for sorting. For example, the identified records may correspond to a particular patient. In some examples, a model may be generated based on records for more than one patient, such as records for a group of patients with similar demographic characteristics (e.g., age, weight, medical history, and so on). One or more clustering algorithms (e.g., k-means, density-based, centroid-based, etc.) can be employed to identify similar patients. In some cases, records older than a predetermined number of hours, days, weeks, etc. may be excluded.

At block 510, a set of attributes may be identified as available for use in sorting. For example, the data store 400 may include a flag for each attribute indicating whether the attribute is “sortable.” Additionally or alternatively, a selection of attributes (and in some cases, a sorting order) may be obtained as user input. For example, a user may specify that the records should first be sorted into buckets according to sensor glucose rate of change, then records in each bucket should be sorted into sub-buckets according to sensor glucose value, then into further sub-buckets according to the day of the week, and so on.

At block 515, an attribute may be selected for use in sorting. For example, the selected attributed may be a randomly selected attribute from the identified set of attributes, the next attribute specified by a user, etc.

At block 520, buckets are created for the attribute, each bucket corresponding to a single value for the attribute or a range of values for the attribute. For example, if the attribute corresponds to a discrete set of values (e.g., day of the week, monitoring device alert conditions, etc.) the component can create a single bucket for each discrete value. As another example, if the attribute corresponds to a continuous range of values, the component may identify the largest and smallest values for the selected attribute in the identified set of records, split that range into a predetermined number of sub-ranges (e.g., 4, 10, 50, 1000), and assign each sub-range to a bucket. In some cases, the range of values may be split evenly (or nearly evenly) into a predetermined number of buckets (e.g., 5, 20, 100) so that each sub-range includes the same (or close to the same) number of records.

At block 525, the records are assigned to the created buckets based on the selected attribute. If there are other attribute available for selection, block 525 returns to block 515; otherwise, block 525 proceeds to block 530.

Blocks 530-550 correspond to a loop through the buckets to determine whether each bucket should be further bucketized (i.e., sub-divided). At decision block 535, if the number of records in the bucket is greater than a predetermined threshold, then block 535 proceeds to block 540; otherwise block 535 proceeds to block 545. In this example, the component uses the number of records in a bucket as a termination condition. One of ordinary skill in the art will recognize that other termination conditions may be used, such as a maximum number of levels of buckets (sub-buckets) to generate, a lack of variation between values for any remaining attributes, and so on.

At block 540, the currently selected bucket is subdivided into smaller buckets. Block 540 returns to block 535 to check for the termination condition.

At block 545, an average bias value is calculated and stored for the records in the bucket based on the bias attribute values associated with each record. For example, the component may calculate the mean (e.g., arithmetic or geometric), median, or mode of the records in the bucket. In some cases, the component may calculate a weighted average based on the age of each record (or the sensor health/age) such that older records (or records generated by less reliable sensors) are given less weight that newer records (or records generated by more reliable sensors) and records more than a certain age are ignored or excluded altogether.

At block 550, if there are any buckets left to analyze, block 550 loops back to block 530 to select the next bucket. Otherwise, block 550 proceeds to block 555.

At block 555, the generated buckets are stored as a model in association with the patient (or group of patients). The model can be used to estimate a bias value that increases accuracy when calculating an estimated blood glucose level for the patient (or group of patients).

One of ordinary skill in the art will recognize that a “bucket” can be any one or more types of data structures, such as an array, list, and so on. Furthermore, and as discussed above, any number of techniques can be used to generate a model for estimating bias values in conjunction with or in place of the bucketization techniques described above.

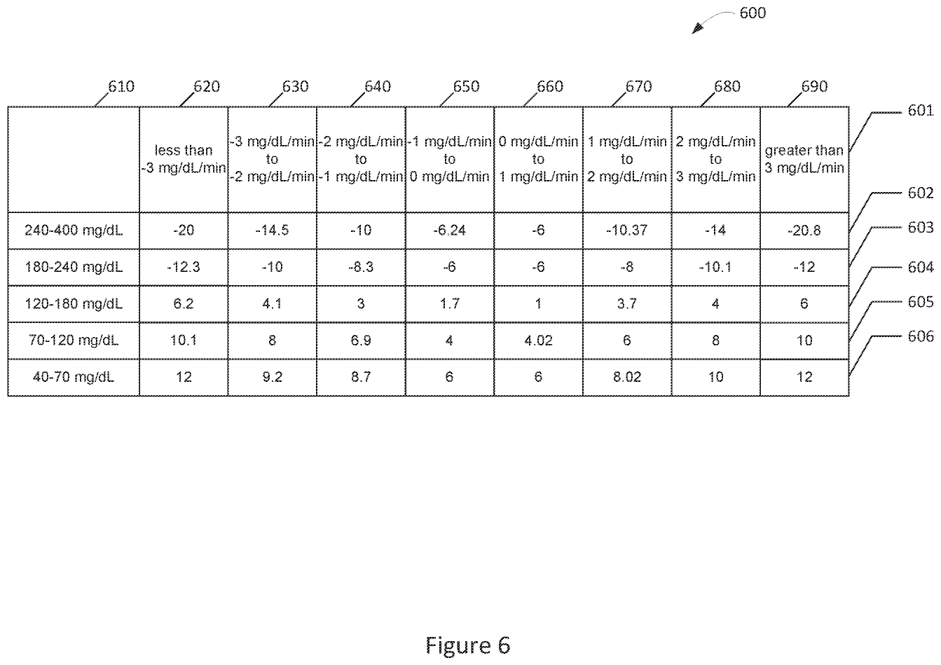

FIG. 6 is a block diagram illustrating an example predictive model 600 for estimating bias values, in accordance with some embodiments of the disclosed technology. In this example, the records have been bucketized according to two attributes—sensor glucose rate of change (each bucket represented with a label in row 601) and sensor glucose value (each bucket represented with a label column 610). The intersections of each of rows 602-606 and columns 620-690 represent the bias values corresponding to the records with attribute values that fall within the corresponding ranges for SG value and SG rate of change. For example, the intersection of row 605 and column 670 represents the bucket (or sub-bucket) that contains the records that have a sensor glucose value between 70 mg/dL and 120 mg/dL and that have a sensor glucose rate of change between 1 mg/dL/min and 2 mg/dL/min. As indicated in FIG. 6, these records have an associated estimated bias value of 6. As another example, the intersection of row 606 and column 690 represents the bucket (or sub-bucket) that contains the records that have a sensor glucose value between 40 mg/dL and 70 mg/dL and that have a sensor glucose rate of change greater than 3 mg/dL/min. As indicated in FIG. 6, these records have an associated estimated bias value of 12. Thus, the predictive model 600 can be consulted (e.g., when an insulin dosage is being calculated for a patient) based on the patient's current sensor glucose value and sensor glucose rate of change to determine a bias value. This bias value can be used to adjust (e.g., can be added to) the patient's current sensor glucose value to generate a more accurate estimate for the patient's blood glucose level. One skilled in the art will appreciate that while FIG. 6 provides an illustration that is easily comprehensible by a human reader, the actual information may be stored using different data structures and data organizations. For example, while a two-dimensional model is illustrated, a model of any number of dimensions may be generated by further subdividing records into additional buckets based on additional attributes. Moreover, one of ordinary skill in the art will recognize that several types of models can include different types of data. For example, a regression model can include a number of coefficients used to calculate bias values. Additionally or alternatively, if the model is a neural network, the neural network can include a set of weights for associated activation functions.

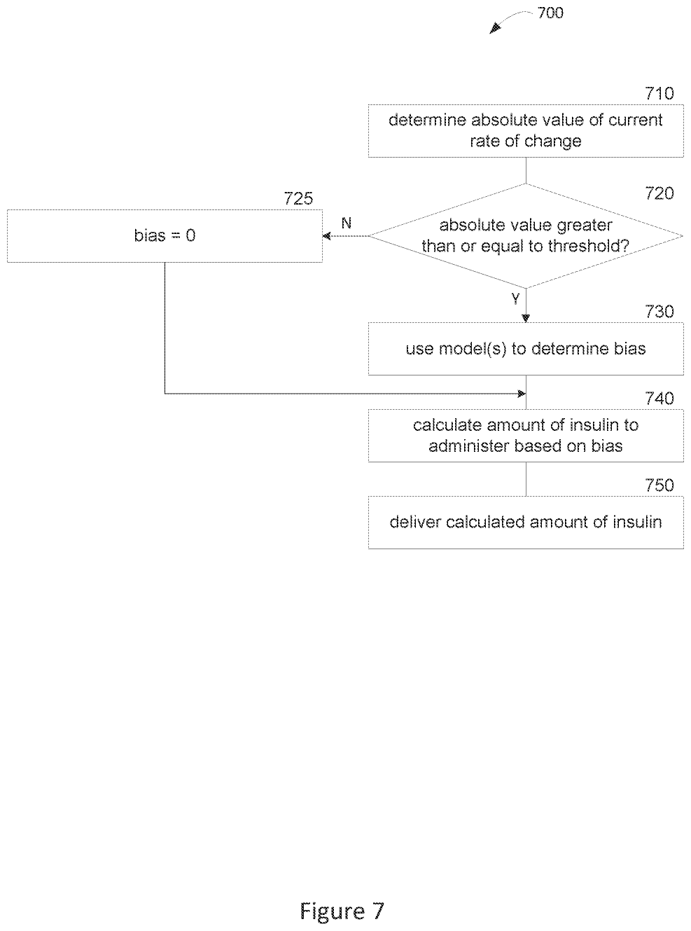

FIG. 7 is a flow diagram illustrating example techniques 700 for estimating bias values based on applying a predictive model, in accordance with some embodiments of the disclosed technology. Although the example of FIG. 7 depicts bias value estimation in the context of insulin delivery, it should be appreciated that this context is merely provided as an example and that bias value estimation is not limited to this context.

At block 710, the absolute value of the patient's current sensor glucose rate of change is determined. For example, if the current SG rate of change is-3 mg/dL/min, the absolute value would be 3.

At decision block 720, if the absolute value of the patient's current sensor glucose rate of change is greater than or equal to a predetermined threshold (e.g., 0.0 mg/dL/min, 0.1 mg/dL/min, 0.9 mg/dL/min), block 720 proceeds to at block 730. Otherwise, block 720 proceeds to block 725. In this manner, the threshold can be used to determine whether or not to apply a bias value. For example, if the threshold has a non-zero value (e.g., 0.1 mg/dL/min), the threshold can enable a SG value exhibiting a low rate of change to avoid adjustment with a bias value.

At block 725, the bias value is set to 0 if the absolute value does not violate the threshold. Block 725 proceeds to block 740.

At block 730, one or more models are used to determine a bias value if the absolute value violates the threshold. For example, if the model is a bucketized model, the buckets can be traversed to find the bucket corresponding to the patient's current condition and current contextual information to determine a bias value based on that bucket, such as the average, mean, median, or mode bias value for that bucket. As another example, if the model is a machine learning model or regression model, the model or regression can be applied to the current patient and contextual information to determine a bias value.

At block 740, an insulin dosage (e.g., a meal bolus dosage) is calculated for the patient based on the bias value. For example, an estimated blood glucose value may be calculated by adding the bias value to a sensor glucose value. This estimated blood glucose value can be used in place of an actual blood glucose level in a formula for calculating insulin dosage. One of ordinary skill in the art will recognize that the disclosed techniques can be used in conjunction with any techniques for calculating an insulin dosage that is based on a blood glucose level or a sensor glucose value.

At block 750, the calculated insulin dosage is delivered to the patient. Block 750 may be performed by one or more of devices 102-106 and/or system 108. For example, device 106 may cause delivery by device 102 based on communicating the dosage to device 102 or a user of device 102.

Although the techniques disclosed herein may facilitate more accurate insulin dosages, it should be appreciated that the techniques have wider applicability. For example, SG values that have been adjusted for bias can be presented to a user of a CGM, thereby enabling the user to be better informed when making glucose management decisions (e.g., what to cat, when to cat, and/or which exercise regimen to undertake).

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. For example, although the above techniques are generally described in the context of estimating blood glucose levels, one of ordinary skill in the art will recognize that these techniques can be used to estimate other analyte levels in other fluids where there may be a bias or difference between an estimated value and an actual value. Like reference numerals may refer to like elements throughout the description of the figures.

Any of the herein described techniques, operations, methods, programs, algorithms, or codes may be converted to, or expressed in, a programming language or computer program embodied on a computer, processor, or machine-readable medium. The terms “programming language” and “computer program,” as used herein, each include any language used to specify instructions to a computer or processor, and include (but is not limited to) the following languages and their derivatives: Assembler, Basic, Batch files, BCPL, C, C+, C++, Delphi, Fortran, Java, JavaScript, machine code, operating system command languages, Pascal, Perl, PLI, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. To the extent consistent, any or all of the aspects detailed herein may be used in conjunction with any or all of the other aspects detailed herein. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

While several embodiments of the disclosure have been depicted in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

I/We claim:

1. A system for estimating blood glucose with increased accuracy, the system comprising:
one or more processors; and
one or more processor-readable media storing instructions which, when executed by the one or more processors, cause performance of:
obtaining a sensor glucose value;
calculating a sensor glucose rate of change based on the sensor glucose value;
determining an estimated glucose bias value corresponding to the sensor glucose rate of change based on applying, to the sensor glucose rate of change, a predictive model for estimating glucose bias values corresponding to differences between actual blood glucose levels and contemporaneous sensor glucose values; and
adjusting the sensor glucose value based on accounting for the estimated glucose bias value.

2. The system of claim 1, wherein the one or more processor-readable media further store instructions which, when executed by the one or more processors, cause performance of:
calculating an insulin dosage based on the adjusted sensor glucose value; and
causing delivery of the insulin dosage based on communicating the insulin dosage to an insulin delivery device or a user of the insulin delivery device.

3. The system of claim 1, wherein the one or more processor-readable media further store instructions which, when executed by the one or more processors, cause performance of:
obtaining contextual information indicative of a context in which the sensor glucose value was generated.

4. The system of claim 3, wherein the contextual information includes at one of a group comprising a time of day, a day of week, a sensor age, a sensor condition descriptor, and an indication of any active alerts.

5. The system of claim 3, wherein applying the predictive model comprises applying the predictive model to the sensor glucose rate of change and the contextual information, and wherein the estimated glucose bias value corresponds to the sensor glucose rate of change and the contextual information.

6. The system of claim 1, wherein determining the estimated glucose bias value comprises selecting a bias value from a plurality of candidate bias values, each candidate bias value corresponding to a respective probability value.

7. The system of claim 1, wherein the one or more processor-readable media further store instructions which, when executed by the one or more processors, cause performance of:
presenting the adjusted sensor glucose value to a user.

8. A processor-implemented method for estimating blood glucose with increased accuracy, the method comprising:
obtaining a sensor glucose value;
calculating a sensor glucose rate of change based on the sensor glucose value;
determining an estimated glucose bias value corresponding to the sensor glucose rate of change based on applying, to the sensor glucose rate of change, a predictive model for estimating glucose bias values corresponding to differences between actual blood glucose levels and contemporaneous sensor glucose values; and
adjusting the sensor glucose value based on accounting for the estimated glucose bias value.

9. The method of claim 8, further comprising:
calculating an insulin dosage based on the adjusted sensor glucose value; and
causing delivery of the insulin dosage based on communicating the insulin dosage to an insulin delivery device or a user of the insulin delivery device.

10. The method of claim 8, further comprising:
obtaining contextual information indicative of a context in which the sensor glucose value was generated.

11. The method of claim 10, wherein the contextual information includes at one of a group comprising a time of day, a day of week, a sensor age, a sensor condition descriptor, and an indication of any active alerts.

12. The method of claim 10, wherein applying the predictive model comprises applying the predictive model to the sensor glucose rate of change and the contextual information, and wherein the estimated glucose bias value corresponds to the sensor glucose rate of change and the contextual information.

13. The method of claim 8, wherein determining the estimated glucose bias value comprises selecting a bias value from a plurality of candidate bias values, each candidate bias value corresponding to a respective probability value.

14. The method of claim 8, further comprising:
presenting the adjusted sensor glucose value to a user.

15. One or more processor-readable media storing instructions which, when executed by one or more processors, cause performance of:
obtaining a sensor glucose value;
calculating a sensor glucose rate of change based on the sensor glucose value;
determining an estimated glucose bias value corresponding to the sensor glucose rate of change based on applying, to the sensor glucose rate of change, a predictive model for estimating glucose bias values corresponding to differences between differences between actual blood glucose levels and contemporaneous sensor glucose values; and
adjusting the sensor glucose value based on accounting for the estimated glucose bias value.

16. The one or more processor-readable media of claim 15, further storing instructions which, when executed by the one or more processors, cause performance of:
calculating an insulin dosage based on the adjusted sensor glucose value; and
causing delivery of the insulin dosage based on communicating the insulin dosage to an insulin delivery device or a user of the insulin delivery device.

17. The one or more processor-readable media of claim 15, further storing instructions which, when executed by the one or more processors, cause performance of:
obtaining contextual information indicative of a context in which the sensor glucose value was generated.

18. The one or more processor-readable media of claim 17, wherein the contextual information includes at one of a group comprising a time of day, a day of week, a sensor age, a sensor condition descriptor, and an indication of any active alerts.

19. The one or more processor-readable media of claim 17, wherein applying the predictive model comprises applying the predictive model to the sensor glucose rate of change and the contextual information, and wherein the estimated glucose bias value corresponds to the sensor glucose rate of change and the contextual information.

20. The one or more processor-readable media of claim 15, wherein determining the estimated glucose bias value comprises selecting a bias value from a plurality of candidate bias values, each candidate bias value corresponding to a respective probability value.
